SEQUENCE LISTING (34 sequences)

SEQ ID NO: 1 - DNA, 5 bp, Artificial Sequence
  Synthetic construct
ttatt

SEQ ID NO: 2 - DNA, 22 bp, Artificial Sequence
  Synthetic construct
caaggagctcgtcgtcattttc

SEQ ID NO: 3 - DNA, 28 bp, Artificial Sequence
  Synthetic construct
actcctctcagctctcattcctatcaac

SEQ ID NO: 4 - DNA, 27 bp, Artificial Sequence
  Synthetic construct
aagatgagcaaggcgtgtaggtgtggc

SEQ ID NO: 5 - DNA, 30 bp, Artificial Sequence
  Synthetic construct
cgtcccttcttctttaccttctcctggctc

SEQ ID NO: 6 - DNA, 25 bp, Artificial Sequence
  Synthetic construct
cgtgtcaccagccattgagtgtcac

SEQ ID NO: 7 - DNA, 25 bp, Artificial Sequence
  Synthetic construct
cccctggaagcatcgctggttttat

SEQ ID NO: 8 - DNA, 28 bp, Artificial Sequence
  Synthetic construct
aagcagcttgaagacggtcgaactctgg

SEQ ID NO: 9 - DNA, 29 bp, Artificial Sequence
  Synthetic construct
aagagtcactcacttttgacagttatacg

SEQ ID NO: 10 - DNA, 25 bp, Artificial Sequence
  Synthetic construct
atgtgtagggcacagaagtcacctg

SEQ ID NO: 11 - DNA, 27 bp, Artificial Sequence
  Synthetic construct
cgcatgttatcacaggaagtcgtgttg

SEQ ID NO: 12 - DNA, 26 bp, Artificial Sequence
  Synthetic construct
tgtcaacactctcccaggtagtaacg

SEQ ID NO: 13 - DNA, 26 bp, Artificial Sequence
  Synthetic construct
tccatcgccacctaattctctttcct

SEQ ID NO: 14 - DNA, 26 bp, Artificial Sequence
  Synthetic construct
ggtacaaatctcaggtccctattgga

SEQ ID NO: 15 - DNA, 6 bp, Artificial Sequence
  Synthetic construct
aacctc

SEQ ID NO: 16 - DNA, 20 bp, Artificial Sequence
  Synthetic construct
cactcccttctgacacttat

SEQ ID NO: 17 - DNA, 27 bp, Artificial Sequence
  Synthetic construct
aacagtcactcagcattttgacaatag

SEQ ID NO: 18 - RNA, 41 nt, Artificial Sequence
  Synthetic construct
uaauuucuacuaaguguagaucccccagcgcuucagcguuc

SEQ ID NO: 19 - RNA, 41 nt, Artificial Sequence
  Synthetic construct
uaauuucuacuaaguguagaugugguauucuugcuaguuac

SEQ ID NO: 20 - DNA, 18 bp, Artificial Sequence
  Synthetic construct
ccgacgacgactactagc

SEQ ID NO: 21 - DNA, 23 bp, Artificial Sequence
  Synthetic construct
agagtaaacgtaaaaagaaggtt

SEQ ID NO: 22 - DNA, 18 bp, Artificial Sequence
  Synthetic construct
aacacaagctttcggcag

SEQ ID NO: 23 - DNA, 22 bp, Artificial Sequence
  Synthetic construct
gaaatttggatctttgtcatcc

SEQ ID NO: 24 - DNA, 27 bp, Artificial Sequence
  Synthetic construct
atctcgtcactctctctctctgacgtg

SEQ ID NO: 25 - DNA, 28 bp, Artificial Sequence
  Synthetic construct
aacgtcactctctctctctcttgacgag

SEQ ID NO: 26 - DNA, 28 bp, Artificial Sequence
  Synthetic construct
ctctctctctagtcactgaatgacttta

SEQ ID NO: 27 - DNA, 27 bp, Artificial Sequence
  Synthetic construct
ctctctctctagtcacatgagtgactt

SEQ ID NO: 28 - DNA, 27 bp, Artificial Sequence
  Synthetic construct
atatatatatcgtcaatgaatgacgtc

SEQ ID NO: 29 - DNA, 18 bp, Artificial Sequence
  Synthetic construct
cggtggacaaattgtcac

SEQ ID NO: 30 - DNA, 22 bp, Artificial Sequence
  Synthetic construct
cttctctggatttaacacactt

SEQ ID NO: 31 - RNA, 45 nt, Artificial Sequence
  Synthetic construct
uaauuucuacuaaguguagauuuugugugcugacucuaucauuau

SEQ ID NO: 32 - DNA, 27 bp, Artificial Sequence
  Synthetic construct
tactcgtcacacacacacactgacgtg

SEQ ID NO: 33 - DNA, 27 bp, Artificial Sequence
  Synthetic construct
atgtcgtcagtgtgtgtgtgtgacgtg

SEQ ID NO: 34 - DNA, 27 bp, Artificial Sequence
  Synthetic construct
tagtcgtcagagagagagagtgacgtg

(19) United States
(12) Patent Application Publication
(10) Pub. No.: US 2024/0093272 A1
(43) Pub. Date: Mar. 21, 2024

(54) CAS12 DETECTION METHODS AND COMPOSITIONS

(71) Applicant: The New Zealand Institute for Plant and Food Research Limited, Auckland (NZ)

(72) Inventor: Revel Scott MacGregor DRUMMOND, Auckland (NZ)

(73) Assignee: The New Zealand Institute for Plant and Food Research Limited, Auckland (NZ)

(21) Appl. No.: 18/255,268

(22) PCT Filed: Dec. 1, 2021
(86) PCT No.: PCT/IB2021/061154

§ 371 Date: May 31, 2023

(30) Foreign Application Priority Data
Dec. 1, 2020 (NZ) 770539

(51) Int. Cl. C12Q 1/6818; C12Q 1/6823; C12Q 1/6876

(52) U.S. Cl. CPC C12Q 1/6818; C12Q 1/6823; C12Q 1/6876; C12Q 1/6844

(57) ABSTRACT

Provided is a Cas12 reporter oligonucleotide comprising an arm-stem-loop-stem-arm structure, having a fluorophore molecule on one end of the oligonucleotide and a quencher molecule on the other end, wherein the oligonucleotide is able to be cleaved by Cas12 enzyme in the presence of a guide RNA bound to a target sequence. Also provided include kits and methods of detecting the presence or absence of a target sequence.

### FIELD

The disclosures provided herein relate to the field of molecular diagnostics and specifically to a reporter oligonucleotide for CRISPR-Cas nucleic acid detection methods.

### BACKGROUND

Current molecular diagnostics suffer from a number of challenges, including some combination of slow development, low-throughput, labour-intensity, and expense of equipment and labour, as summarized in the table below.

TABLE 1

| | Visual | | | Real-Time |
|---|---|---|---|---|
| Feature | assessment | ELISA | PCR | PCR |
| Development | + | X | + | + |
| time | | | | |
| High- | X | + | X | + |
| throughput | | | | |
| Specificity | X | X | + | + |
| Sensitivity | X | X | + | + |
| Cost | X | + | X | X |
| Field | + | X | X | X |
| applicability | | | | |
| Before/No | X | + | + | + |
| symptoms | | | | |

Since its initial discovery, at least 6 types and 22 subtypes of CRISPR-Cas systems have been discovered and explored. Diagnostic CRISPR systems are most often derived from types II, V, and VI. Different types of CRISPR-Cas systems which have been identified in different microorganisms can target DNA (e.g. Cas9 and Cas12 enzymes; FIGS. 1A and 1B) or RNA (e.g. Cas13 enzyme). Viral, bacterial, and non-infectious diseases such as cancer can all be diagnosed using the extraordinarily specific targeting activity of CRISPR enzymes.

Diagnostic tests using Cas12 and Cas13 enzymes have been developed. Cas13a is a single-component, RNA-guided and targeting enzyme, which is specific for ssRNA and collaterally cleaves nearby non-target RNAs. In contrast, Cas12a is an RNA-guided, DNA-targeting enzyme which targets DNA and collaterally cleaves ssDNA (Li et al. (2018) Cell Discov. 4:1-4; Chen et al. (2018) Science 360:436-439; FIG. 1C). Many of the Cas13 subtypes (and some Cas12) have different base preferences, cleaving at specific dinucleotide motifs (Gootenberg et al. (2018) Science 360:439). Different platforms have been developed to exploit the collateral cleavage activity by these two proteins. Specific high sensitivity enzymatic reporter unlocking (SHERLOCK) was introduced in 2017, which exploits Cas13a for the detection of RNA molecules and a diagnostic platform based on this method was developed in 2018 (Gootenberg et al. (2017) Science 356:438-442; Gootenberg et al. (2018) Science 360:439). At the same time, a Cas-12a-based diagnostic tool called one-hour low-cost multipurpose highly efficient system (HOLMES) was introduced in 2017 and a diagnosis platform was soon described (Cheng (2017) CN107488710; Li et al. (2018) Cell Disc. 4:20). Chen et al. (2018) also describes a Cas12-based detection system, DNA Endonuclease Targeted CRISPR Trans Reporter (DETECTR) (Science 10:1126). Conveniently, these methods allow for direction the Cas enzyme to a target sequence using a guide RNA, cleavage of the target sequence, and collateral cleavage of nearby reporter nucleotides that are detectable upon cleavage. In these methods, a fluorophore-quencher (FQ) reporter oligonucleotide is used to measure the collateral cleavage activity, typically composed of a fluorophore (F) and a quencher (Q) connected by a short 5-8 nucleotide sequence.

Current CRISPR-Cas based methods involve several steps, expensive kits and equipment (e.g., extraction and amplification kits, magnetic bead separator or centrifuge, thermocycler), and risk carryover contamination. For example, the method can involve nucleic acid isolation from a sample, moving the nucleic acids to an amplification reaction, amplification involving high temperature (e.g., by PCR), either opening the amplification vessel to add the CRISPR-Cas reagents, or transferring the nucleic acids to a separate vessel for cleavage and detection.

It is an object of the present invention to streamline current CRISPR-Cas detection methods and avoid carryover contamination.

### SUMMARY OF THE INVENTION

Provided herein are methods, kits, and compositions for specific target nucleic acid detection using Cas12 collateral cleavage to cleave a dual labelled reporter oligonucleotide. In particular, a Cas12 reporter oligonucleotide comprising: a) at least 25 nucleotides; b) an arm-stem-loop-stem-arm structure, wherein: the arm regions are single-stranded, the stem regions hybridize to one another, with base pairing extending over 4 or more nucleotides, and the loop region is single-stranded and greater than 5 nucleotides in length; c) at least 2 Cas12 preferred dinucleotides, wherein the Cas12 preferred dinucleotides are in the loop and/or arm regions; and d) a fluorophore molecule on one end of the oligonucleotide and a quencher molecule on the other end; wherein the oligonucleotide is able to be cleaved by Cas12 enzyme in the presence of a guide RNA bound to a target sequence. In some embodiments, the oligonucleotide is cleaved by the Cas12 enzyme in the presence of the guide RNA bound to target sequence.

Cleavage of the reporter oligonucleotide by Cas12 occurs more quickly as the number of Cas12 preferred dinucleotides increases. In some embodiments, the reporter oligonucleotide comprises at least 2, 3, 4, 5, 6, 7, 8, 9, 10, 11, 12, 13, 14, 15 or more Cas12 preferred dinucleotides. In some embodiments, the Cas12 preferred dinucleotides are CT and TC. In some embodiments the Cas12 preferred dinucleotides are AT and TA, GT and TG, and CA and AC. In some embodiments, the reporter oligonucleotide has no dinucleotide preference.

In some embodiments, one of the arm regions has 6-14 nucleotides, and the Cas12 preferred dinucleotides are in the arm. In some embodiments, the loop region has 8-14 nucleotides, and the Cas12 preferred dinucleotides are in the loop.

The structure of the reporter oligonucleotide is such that the dinucleotides preferred by the Cas12 are present in the available, single-stranded part of the oligonucleotide, ensuring that collateral cleavage can occur efficiently.

In some embodiments, the Cas12 enzyme is Cas12a, Cas12b, Cas12c, Cas12d, or Cas12e. In some embodiments, the Cas12 enzyme is selected from the group consisting of AapCas12a, AacCas12b, CmeCas12a, FnCas12a, Engen® LbaCas12a (NEB), or YmeCas12a (NEB). In some embodiments, the Cas12 enzyme is thermotolerant, e.g., AapCas12a, AacCas12b, YmeCas12a, CmeCas12a, and Engen® LbaCas12a (NEB).

In some embodiments, the Cas12 enzyme is CmeCas12a, YmeCas12a, or LbaCas12a. In some embodiments, the Cas12 enzyme is CmeCas12a and the Cas12 preferred dinucleotides are CT and TC. In some embodiments, the Cas12 enzyme is LbaCas12a.

Various combinations of arm, stem and loop region lengths are possible within the parameters described above, that result in formation of an oligonucleotide secondary structure that brings the fluorophore and quencher into sufficient proximity to quench fluorescence. In some embodiments, the fluorophore and quencher are within about 50 angstroms or less of each other. In some embodiments, the fluorophore and quencher are within about 40 angstroms or less of each other. For example, in some cases the fluorophore and quencher are within about 34 angstroms or less, about 30 angstroms or less, about 27 angstroms or less, about 24 angstroms or less, about 20 angstroms or less, about 17 angstroms or less, or about 15 angstroms or less of each other.

In some embodiments, the Cas12 reporter oligonucleotide further comprises an internal quencher. In some embodiments, the internal quencher is positioned close enough to the fluorophore molecule to effectively quench signal (in combination with the other non-internal quencher), whether the proximity is through linear or secondary structure. In some embodiments, at least 2 Cas12 preferred dinucleotides are present between the fluorophore and internal quencher, e.g., 2, 3, 4, 5, 6, 7, 8, or more Cas12 preferred dinucleotides. In some embodiments, the internal quencher is placed 5, 6, 7, 8, 9, 10, 11, 12, or more nucleotides (or about 17, about 20, about 24, about 27, about 30, about 34, about 37, or about 40 angstroms) away from the fluorophore molecule. In some embodiments, the internal quencher is ZEN and the quencher (non-internal) is Iowa Black FQ. In some embodiments, the internal ZEN quencher is incorporated between the 9th and 10th nucleotides away from the fluorophore molecule. In some embodiments, the internal ZEN quencher is incorporated between 25-30 angstroms away from the fluorophore molecule.

In some embodiments, the Cas12 reporter oligonucleotide comprises a sequence that is at least 75, 80, 81, 82, 83, 84, 85, 86, 89, 90, 91, 92, 93, 94, 95, 96, 97, 98, 99 or 100% identical to a sequence selected from the group consisting of: SEQ ID NO:1-17 and 24-28. In some embodiments, the Cas12 reporter oligonucleotide comprises a sequence that is at least 90% identical to a sequence selected from the group consisting of:

(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

In some embodiments, the Cas12 reporter oligonucleotide comprises a sequence selected from the group consisting of:

(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

In some embodiments, the Cas12 reporter oligonucleotide is a sequence selected from the group consisting of:

(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

Further provided are kits, wherein the kit comprises: a) a Cas12 reporter oligonucleotide as described herein; b) a nucleotide polymerase; and c) a thermotolerant Cas12 enzyme. In some embodiments, the nucleotide polymerase is a DNA and/or RNA polymerase. In some embodiments, the nucleotide polymerase is Bst2.0 and optionally reverse transcriptase. In some embodiments, the nucleotide polymerase is Q5U and optionally reverse transcriptase. In some embodiments, the kit further comprises primers specific for a target sequence. In some embodiments, the kit further comprises a guide RNA specific for a target sequence. In some embodiments, the kit further includes at least one buffer, dNTPs, or other reagents for amplification and/or Cas12 activity. Also provided are methods for detecting a target sequence. In some embodiments, the method comprises: a) isolating target nucleic acid; b) contacting the target nucleic acid with: a nucleotide polymerase, oligonucleotide primers specific for a target sequence on the target nucleic acid, a guide RNA oligonucleotide specific for the target sequence, a thermotolerant Cas12 enzyme, and a Cas12 reporter oligonucleotide as described herein; c) incubating at a temperature of about 35 C to about 70 C, for example about 37 C to about 60 C, for at least 10 minutes; and d) detecting fluorescence when the oligonucleotide is cleaved by the Cas12 enzyme. In some embodiments, the method comprises a) isolating target nucleic acid; b) contacting the target nucleic acid with: a nucleotide polymerase, oligonucleotide primers specific for a target sequence on the target nucleic acid, a guide RNA oligonucleotide specific for the target sequence, a thermotolerant Cas12 enzyme, and the Cas12 reporter oligonucleotide as described herein; c) incubating at a temperature of about 35 C to about 70 C, e.g., about 37 C to about 60 C, for at least 10 minutes; and d) measuring fluorescence, wherein detecting fluorescence produced by cleavage of the oligonucleotide by the Cas12 enzyme is indicative of the presence of the target sequence, and wherein the absence of fluorescence is indicative of the absence of the target sequence. In some embodiments, the incubating is at a temperature of about 37 C, about 42 C, about 45 C, about 48 C, about 50 C, about 52 C, about 55 C, about 58 C, or about 60 C. In some embodiments, the incubating is at least 15, 30, 45, or 60 minutes. In some embodiments, the nucleotide polymerase is Bst2.0. In some embodiments, reverse transcriptase is also included. In some embodiments, the method comprises a) isolating target nucleic acid; b) contacting the target nucleic acid with a nucleotide polymerase and oligonucleotide primers specific for a target sequence on the target nucleic acid, and amplifying the target sequence to produce amplified target sequence; c) contacting the amplified target sequence with a guide RNA oligonucleotide specific for the target sequence, a thermotolerant Cas12 enzyme, and the Cas12 reporter oligonucleotide as described herein; d) incubating at a temperature of about 35 C to about 70 C, for example about 37 C to about 60 C, for at least 15 minutes; and e) detecting fluorescence when the oligonucleotide is cleaved by the Cas12 enzyme. In some embodiments, the method comprises a) isolating target nucleic acid; b) contacting the target nucleic acid with a nucleotide polymerase and oligonucleotide primers specific for a target sequence on the target nucleic acid, and amplifying the target sequence to produce amplified target sequence; c) contacting the amplified target sequence with a guide RNA oligonucleotide specific for the target sequence, a thermotolerant Cas12 enzyme, and the Cas12 reporter oligonucleotide as described herein; d) incubating at a temperature of about 35 C to about 70 C, e.g., about 37 C to about 60 C, for at least 10 minutes; and e) measuring fluorescence, wherein detecting fluorescence produced by cleavage of the oligonucleotide by the Cas12 enzyme is indicative of the presence of the target sequence, and wherein the absence of fluorescence is indicative of the absence of the target sequence. In some embodiments, the incubating is at a temperature of about 37 C, about 42 C, about 45 C, about 48 C, about 50 C, about 52 C, about 55 C, about 58 C, or about 60 C. In some embodiments, the incubating is at least 15, 30, 45, or 60 minutes. In some embodiments, the nucleotide polymerase is QSU, Taq, or a Taq derivative. In some embodiments, reverse transcriptase is also included.

In some embodiments, the thermotolerant Cas12 enzyme is CmeCas12a, YmeCas12a, or LbaCas12a. In some embodiments, the isolating is carried out using enzymatic purification.

In some embodiments, the methods can be multiplexed to detect more than one target sequence. In that case, at least a first guide RNA that specifically hybridizes to a first target sequence and a second guide RNA that specifically hybridizes to a second target sequence are used. The level of multiplexing can be increased, e.g, to detect 2, 3, 4, 5, 6, 7, 8 or more target sequences. One Cas12 reporter oligonucleotide can be used so that a single signal (e.g., a single wavelength) is detected upon cleavage, regardless of how many, or which, target sequence is present. Such multiplexing can be useful, e.g., to detect multiple target sequences from a single pathogen, or multiple mutations linked to a single disorder. Alternatively, more than one Cas12 reporter oligonucleotide can be used, e.g., with different fluorophore and quencher pairs. In such cases, each Cas12 reporter oligonucleotide can be designed to have the preferred dinucleotides of a different Cas12 enzyme. For example, a first Cas12 reporter oligonucleotide can be designed with a first fluorophore/quencher pair and multiple CT or TC dinucleotides for cleavage by CmeCas12a, and a second Cas12 reporter oligonucleotide can be designed with a second fluorophore/quencher pair and multiple alternative preferred dinucleotides for cleavage by a different Cas12a. The activity of each Cas12 enzyme can be targeted to the appropriate target sequence by having each guide RNA targeted to the preferred PAM site of each enzyme on the target sequence.

In some embodiments, the Cas12 reporter oligonucleotide comprises a sequence that is at least 75, 80, 81, 82, 83, 84, 85, 86, 89, 90, 91, 92, 93, 94, 95, 96, 97, 98, 99 or 100% identical to a sequence selected from the group consisting of: SEQ ID NO:1-17 and 24-28. In some embodiments, the Cas12 reporter oligonucleotide comprises a sequence that is at least 90% identical to a sequence selected from the group consisting of:

(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

In some embodiments, the Cas12 reporter oligonucleotide comprises a sequence selected from the group consisting of:

(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

In some embodiments, the Cas12 reporter oligonucleotide is a sequence selected from the group consisting of:

(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

### BRIEF DESCRIPTION OF THE DRAWINGS

FIG. 1 depicts the mode of action for Cas9, Cas12a and the process of CRISPR-DX using Cas12a. FIG. 1A depicts the dsDNA cleavage mechanism of Cas9. TracrRNA: trans-activating crispr RNA; sgRNA: single guide RNA; crRNA: crispr RNA; PAM: protospacer adjacent motif. The target sequence is targeted with the crRNA. The PAM sequence is typically 3-4 nucleotides downstream of the site cut by the Cas9 enzyme.

FIG. 1B depicts the dsDNA cleavage mechanism of Cas12. In contrast to Cas9, Cas12 activity requires a 5′ PAM site and results in a staggered cut of the double stranded target DNA distal to the PAM site. It also results in non-specific collateral cleavage of ssDNA once a sequence specific dsDNA is cleaved.

FIG. 1C shows how the collateral cleavage triggered by Cas12a can be harnessed as a diagnostic tool. The figure is adapted from Li et al. (2019) Trends in Biotechnol. 37:730. The Cas12a effector can both bind and cut ssDNA and dsDNA site-specifically, but the collateral cleavage remains only for ssDNA, indicating that a fully or partially ssDNA reporter is needed. The first step, amplification, is used to produce sufficient dsDNA target for the Cas12a to rapidly bind. In the second step, Transduction, the target DNA is cleaved and the Cas12a left in an active state where it cleaves any single stranded DNA present. In a final step, Reporting, the effects of cleaving the ssDNA reporter molecule are detected. In the example shown the reporting is by the fluorescence of the fluorophore now separated from the quenching moiety. However, the reporter molecules can be changed according to individual needs. LAMP, loop-mediated isothermal amplification; PCR, polymerase chain reaction; RPA, recombinase polymerase amplification; RT, reverse transcription; ssDNA, single stranded DNA.

FIG. 2 shows the predicted secondary structure of the Cas12 reporter oligonucleotides R1 (SEQ ID NO:1), R15 (SEQ ID NO:15), R16 (SEQ ID NO:16), R17 (SEQ ID NO:17), and R19 (SEQ ID NO:24) at 45° C. The R17 and R19 molecules were designed so that the secondary structure brings the fluorophore and quencher into close proximity.

FIG. 3 shows the combined kinetics of the CmeCas12a reaction on the reporter molecules R1-R17 using the E-gene target DNA and guide RNA at 45° C. A no target control (NTC) is included, and the fluorescence data is presented with t=0 fluorescence subtracted. After normalization, the R6, R13 and R17 reporter oligonucleotides had the highest overall cleavage.

FIGS. 4A and 4B show the individual kinetics of the CmeCas12a reactions on the reporter molecules R1-R17 using the E-gene target and guide RNA at 45° C. A no target control (NTC) is included, and the fluorescence data is presented with no normalisation or subtraction. However, the graphs have been scaled such that the y-axis represents the fluorescence of the MBN digested reactions. Longer oligonucleotides with greater numbers of CT and or TC dinucleotides are more rapidly and effectively cleaved by the CmeCas12a enzyme.

FIGS. 5A and 5B show the kinetics of the CmeCas12a reaction on the reporters R1, R6, R7, R9, R12, R13, R16, R17, and R18 (no reporter oligonucleotide) using the E-gene and N-gene targets with the matching guide RNA at 45° C. A no target control (NTC) is included, and the fluorescence data is presented with no normalisation or subtraction. However, in FIG. 5A the graphs have been scaled such that the y-axis represents the fluorescence of the MBN digested reactions and in 5B the x and y-axes have been scaled to show the change in fluorescence over the first 15 minutes of the reaction in greater detail. Longer oligonucleotides with greater numbers of CT and or TC dinucleotides are more rapidly and effectively cleaved by the CmeCas12a enzyme and the target DNA sequence does not markedly alter the effectiveness of the reaction.

FIG. 6 shows the kinetics of LbaCas12a reactions using the R19 reporter molecule on the A, E and N gene targets combined with the appropriate guide RNA. The concentration of the target DNA has been varied from 440 pM to 5.5 pM (with an NTC) to test the limit of detection. The fluorescence data has been collected using an Analytic Jena qPCR machine and is t=0 subtracted. The R19 reporter is effectively cleaved by the LbaCas12a enzyme and the target site does not markedly alter the effectiveness of the reaction.

FIG. 7 shows the kinetics of the LbaCas12a reaction on the reporters R1, R15, R16, R17, R19, R19-CA, R19-GT and R19-GA using the N-gene target and guide RNA at 45° C. A no target control (NTC) is included, and the fluorescence data is presented with no normalisation or subtraction. The greatest rate of reporter cleavage with the lowest background fluorescence is produced from the R19-type reporters.

### DETAILED DESCRIPTION OF THE INVENTION

### Introduction

The present disclosure provides novel methods and compositions for CRISPR-Cas nucleic acid detection. The detection methods rely on collateral cleavage by the Cas12 enzyme, and can be used to detect any nucleic acid sequence. The exact target sequence is generally selected to be near a PAM site (e.g., TTTV for LbaCas12a or TTV for CME Cas12a). The target is determined by the sequence of the Cas12 guide RNA.

The presently described methods can use isothermal amplification and a thermotolerant Cas enzyme, such as Engen® LbaCas12a (NEB), YmeCas12a, or CmeCas12a. This allows for amplification, cleavage, and detection in a single vessel and at a temperature range of about 45-62° C. (e.g. for LAMP amplification) or about 37-42° C. (e.g., for RPA amplification). In addition, an enzymatic nucleic acid isolation step can be employed to reduce the number of reagents and amount of equipment required.

Typically, the Cas enzyme and cleavage reagents are used in a separate vessel from amplification, and the reaction is carried out at a lower temperature. Opening the vessel after an amplification step, however, increases the risk of carryover contamination and the likelihood of a false positive result.

“One-pot” assay systems in which all the reagents required for both amplification and CRISPR-based detection are combined in one pot simplifies the detection procedure and reduces the risk of carryover contamination. However, such systems typically result in a substantial reduction in detection performance and sensitivity compared to two-pot systems, due in part to lower efficiency and higher background fluorescence.

The novel reporter oligonucleotides disclosed herein are effectively and efficiently cleaved by thermotolerant Cas12, allowing the combination of the amplification, cleavage, and detection steps to take place in a single vessel, thus reducing or negating the risk of carryover contamination. These reporter oligonucleotides also successfully reduce or eliminate background fluorescence, which can otherwise significantly affect the detection limit, and thereby extend the detection dynamic range of the methods disclosed herein.

### Definitions

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The terms “a”, “an”, and “the” include plural referents, unless the context clearly indicates otherwise.

The term “comprising” as used in this specification and claims means “consisting at least in part of”. When interpreting statements in this specification, and claims which include the term “comprising”, it is to be understood that other features that are additional to the features prefaced by this term in each statement or claim may also be present. Related terms such as “comprise” and “comprised” are to be interpreted in similar manner.

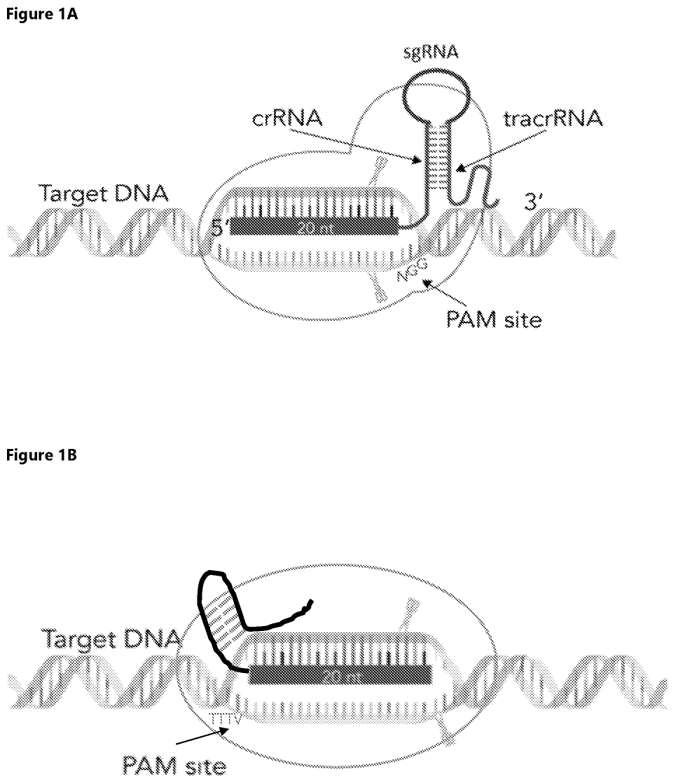

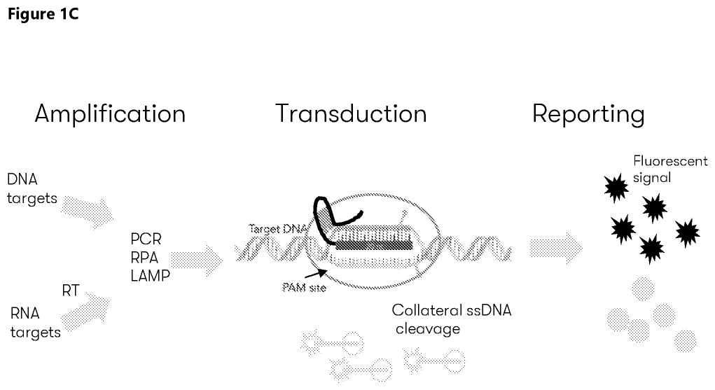

A Cas guide RNA (or CrisprRNA, crRNA) is a sequence specific “probe” that hybridizes to target sequence and guides the Cas enzyme to that sequence for cleavage. Examples of crRNAs are shown in FIGS. 1-3.

A proto-spacer adjacent motif (PAM) sequence is a short sequence (typically 3-8 nucleotides) just outside from the target cleavage site. Each Cas enzyme has a preference for the PAM sequence. Cas12 enzyme PAM sequences are typically T-rich, and several are described in Jacobsen et al. (2020) Nucl. Acid Res. 48:5624.

Collateral cleavage refers to non-sequence specific cleavage of single-stranded DNA by Cas12 enzymes and single-stranded RNA by Cas13 enzymes upon association of the guide RNA with target sequence. This collateral cleavage is exploited in the SHERLOCK, DETECTR, and HOLMES detection techniques.

The terms “isolate”, “purify”, “extract”, and “separate” are not intended to be absolute terms. In the context of the present disclosure, these terms refer to increasing the amount or availability of nucleic acid from a sample, so that the nucleic acid is accessible for enzymatic reactions or other manipulation.

The terms “enzymatic isolation”, “enzymatic purification”, “enzymatic extraction” and “enzymatic separation” refer to release of nucleic acids (or other cellular components) from a cellular or subcellular environment using an enzyme. Appropriate enzymes include RNAGEM (MicroGEM, UK), proteinase K, lyticase, lysozyme, and mutanolysin (see, e.g., Easparro et al. (2016) available at: doi.org/10.1096/fasebj.30.1_supplement.1082.2; Stanton et al. (2019) BioTechniques 66:208-213).

The terms “vessel”, “tube”, “well”, “container”, “vial”, “chamber”, “microchamber” etc. generally refer to a closed container holding one or more reagents or one or more reactions.

The term “nucleotide”, in addition to referring to the naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., hybridization to a complementary base), unless the context clearly indicates otherwise.

The term “nucleic acid” or “polynucleotide” refers to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendent moieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. (Science 254:1497-1500, 1991)). A nucleic acid can be or can include, e.g., a chromosome or chromosomal segment, a vector (e.g., an expression vector), an expression cassette, a naked DNA or RNA polymer, the product of a polymerase chain reaction (PCR), an oligonucleotide, a reporter oligonucleotide, a probe, or a primer. A nucleic acid can be, e.g., single-stranded, double-stranded, or triple-stranded and is not limited to any particular length. Unless otherwise indicated, a particular nucleic acid sequence optionally comprises or encodes complementary sequences, in addition to any sequence explicitly indicated.

The term “oligonucleotide” refers to a nucleic acid that includes at least two nucleic acid monomer units (e.g., nucleotides). An oligonucleotide typically includes from about five to about 175 nucleic acid monomer units, more typically from about eight to about 100 nucleic acid monomer units, and still more typically from about 10 to about 50 nucleic acid monomer units (e.g., about 15, about 20, about 25, about 30, about 35, or more nucleic acid monomer units). The exact size of an oligonucleotide will depend on many factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (Meth. Enzymol. 68:90-99, 1979); the phosphodiester method of Brown et al. (Meth. Enzymol. 68:109-151, 1979); the diethylphosphoramidite method of Beaucage et al. (Tetrahedron Lett. 22:1859-1862, 1981); the triester method of Matteucci et al. (J. Am. Chem. Soc. 103:3185-3191, 1981); automated synthesis methods; or solid support methods; or other methods known to those skilled in the art.

The term “primer” as used herein refers to a polynucleotide capable of acting as a point of initiation of template-directed nucleic acid synthesis when placed under conditions in which polynucleotide extension is initiated (e.g., under conditions comprising the presence of requisite nucleoside triphosphates (as dictated by the template that is copied) and a polymerase in an appropriate buffer and at a suitable temperature or cycle(s) of temperatures (e.g., as in a polymerase chain reaction and RT-LAMP)). To further illustrate, primers can also be used in a variety of other oligonucleotide-mediated synthesis processes, including as initiators of de novo RNA synthesis and in vitro transcription-related processes (e.g., nucleic acid sequence-based amplification (NASBA), transcription mediated amplification (TMA), etc.). A primer is typically a single-stranded oligonucleotide (e.g., oligodeoxyribonucleotide). The appropriate length of a primer depends on the intended use of the primer but typically ranges from 6 to 40 nucleotides, more typically from 15 to 35 nucleotides. Short primer molecules generally require cooler temperatures to form sufficiently stable hybrid complexes with the template. A primer need not reflect the exact sequence of the template but must be sufficiently complementary to hybridize with a template for primer elongation to occur. In certain embodiments, the term “primer pair” means a set of primers including a 5′ sense primer (sometimes called “forward”) that hybridizes with the complement of the 5′ end of the nucleic acid sequence to be amplified and a 3′ antisense primer (sometimes called “reverse”) that hybridizes with the 3′ end of the sequence to be amplified (e.g., if the target sequence is expressed as RNA or is an RNA). A primer can be labelled, if desired, by incorporating a label detectable by spectroscopic, photochemical, biochemical, immunochemical, or chemical means. For the present disclosure, fluorescent dyes (e.g., paired with a quencher) are preferred.

“Hybridization” is an interaction between two usually single-stranded or at least partially single-stranded nucleic acids. Hybridization occurs as a result of base-pairing between nucleobases and involves physicochemical processes such as hydrogen bonding, solvent exclusion, base stacking and the like. Hybridization can occur between fully complementary or partially complementary nucleic acid strands. The ability of nucleic acids to hybridize is influenced by temperature and other hybridization conditions, which can be manipulated in order for the hybridization of even partially complementary nucleic acids to occur. Hybridization of nucleic acids is well known in the art and has been extensively described in Ausubel (Eds.) Current Protocols in Molecular Biology, v. I, II and III (1997).

The terms “thermostable enzyme”, “thermophilic enzyme”, and “thermotolerant enzyme” refers to an enzyme that is stable to heat, is heat resistant and retains sufficient activity after being subjected to elevated temperatures (e.g., 42-60° C.). Examples include Taq and Q5U polymerases, and EnGen® LbaCas12a (NEB), CmeCas12a, YmeCas12a, AacCas12b, AapCas12b enzymes.

The terms “target sequence”, “target region”, “target portion”, “target fragment”, and like terms refer to a region of a target nucleic acid sequence that is to be analysed.

The terms “identical” or “percent identity”, in the context of two or more nucleic acids, or two or more polypeptides, refer to two or more sequences or subsequences that are the same or have a specified percentage of nucleotides, or amino acids, that are the same (e.g., about 60% identity, e.g., at least any of 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising at least about 8-25 amino acids or nucleotides in length, or over a region that is 50-100 amino acids or nucleotides in length, or over the entire length of the reference sequence.

The term “sample” or “biological sample” refers to any composition containing or presumed to contain nucleic acid, e.g., from an individual. The term includes purified or separated components of cells, tissues, or blood, e.g., DNA, RNA, proteins, cell-free portions, or cell lysates. A sample can refer to any type of biological sample, e.g., plant material, fungal material, skin, plasma, serum, whole blood and blood components (buffy coat), saliva, urine, tears, seminal fluid, vaginal fluids, tissue biopsies, and other fluids and tissues, including paraffin embedded tissues. Samples also may include constituents and components of in vitro cultures of cells obtained from an individual, including cell lines.

A “control” sample or value refers to a sample that serves as a reference, usually a known reference, for comparison to a test sample or test conditions. For example, a test sample can be taken from a test condition, e.g., from an individual suspected of having a disease, and compared to samples from known conditions, e.g., from a disease-free individual (negative control), or from an individual known to have the targeted disease (positive control). A control can also represent an average value or a range gathered from a number of tests or results. A control can also be prepared for reaction conditions. For example, a positive control for the presence of nucleic acid could include primers or reagents that will detect a sequence known to be present in the sample, while a negative control would be free of nucleic acids. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “diagnosis” refers to a relative probability that a subject has an infection or disorder such as cancer or a genetic condition. The term is not intended to be absolute, as will be appreciated by any one of skill in the field of medical diagnostics.

The term “kit” refers to any manufacture (e.g., a package or a container) including at least one reagent, such as reagents for nucleic acid extraction, amplification, Cas cleavage, and/or detection as described herein.

### Target Nucleic Acid

As indicated in the Examples below, the present methods and Cas reporter oligonucleotides can be used to detect any target nucleic acid regardless of sequence. The target sequence specificity is governed by the guide RNA, which typically includes 16-30 or 18-25 nucleotides of target-complementary sequence.

The Examples are directed to detection of nucleic acids from SARS-CoV-2. The need for SARS-CoV-2 tests that are rapid, widespread, and able to identify infectious individuals lead us to join the effort to develop strategies for viral RNA detection based on CRISPR technology.

The current gold-standard diagnostic for SARS-CoV-2 infection, quantitative reverse transcription polymerase chain reaction (RT-qPCR), is well established and widely used for screening. Based on primers directed against the nucleocapsid (N), envelope (E), and open reading frame 1ab (ORF1ab) genes, RT-qPCR has an analytical limit of detection (LOD) of 1,000 viral RNA copies/mL (1 copy/μL) (Vogels et al. (2020) Nat. Microbiol. 5:1299). PCR, however, generally requires expensive laboratory equipment.

The presently described methods can be carried out in an isothermal reaction in a high-throughput fashion, resulting in reduced turn-around time, expense, and labour. The simplicity and contained nature of the system also enables the use as a point of care testing process for medium throughput (workplace) and single use (home).

Exemplary target nucleic acids relevant to human health include but are not limited to: viral targets such as influenza, rhinovirus, common cold, HIV, HSV, HPV, hepatitis A-E, chicken pox and shingles, measles, mumps, rubella, herpes, RSV, norovirus, rotovirus, ebola, yellow fever, dengue fever, rabies, meningitis, encephalitis, as well as the SARS viruses. Disease causing bacteria and parasites can also be detected, such as rheumatic fever, gonorrhoea, syphilis, colibacillis, salmonella, staphylococcus, cryptosporidia, and giardia. Disease associated target nucleic acids can be relevant to a number of human disorders, including but not limited to: Parkinson's, Huntington's, and Alzheimer's Diseases; phenylketonuria; cystic fibrosis; sickle cell anemia; hypercholesterolemia; neurofibromatosis; polycystic kidney disease; haemophilia; muscular dystrophy; type I diabetes; hypoparathyroidism. Additional targets are disclosed, for example, at edgar.biocomp.unibo.it and NCBI databases such as omim.org (Online Mendelian Inheritance in Man), ClinVar, Gene, etc.

A number of genetic alleles and mutations associated with cancer can also be detected. Examples include but are not limited to: p53, BRCA1, BRCA2, Her2, EGFR, TP53, Ras, ALK, ROS, RET, FHIT, CDKN, WWOX, PTEN, APC, FANCA, FANCD2, FANCI, HNF1A, MEN1, NSD1, PMS2, RECQL4, RET, SLX4, WRN, and XPC. Additional targets are disclosed, for example, at NCBI databases such as omim.org, ClinVar, Gene, etc.

Exemplary horticultural targets include but are not limited to: plant genotyping (e.g., fruit and other plant sex determinants and plant disease resistance genes); plant virus detection (e.g., grablovirus, grapevine leafroll 3 associated virus); fish sex determinants; plant disease resistance genes; and venturia and other fungi.

Targets relevant to veterinary applications, e.g., animal genotyping, sex determinants, and disease detection, can also be detected using the present methods and compositions.

### Nucleic Acid Isolation and Amplification

Methods for isolating nucleic acids from biological samples are known, e.g., as described in Sambrook, Molecular Cloning: A Laboratory Manual 4th Edition. Several kits are also commercially available (e.g., High Pure RNA Isolation Kit, High Pure Viral Nucleic Acid Kit, and MagNA Pure LC Total Nucleic Acid Isolation Kit from Roche).

Enzymatic methods are available, and can be carried out with minimal equipment and expense. Appropriate enzymes include RNAGEM® (MicroGEM, UK) proteinase K, lyticase, lysozyme, and mutanolysin (see, e.g., Easparro et al. (2016) available at: doi.org/10.1096/fasebj.30.1_supplement.1082.2; Stanton et al. (2019) BioTechniques 66:208-213).

Additional commonly used methods for nucleic acid isolation include guanidinium thiocyanate-phenol-chloroform extraction, Chelex extraction, alkaline extraction, and solid phase (e.g., column or bead) isolation. Such methods are known in the art, and described, e.g., in Ali et al. (2017) Biomed Res. Intl. Art. No. 9306564.

Nucleic acid amplification methods are also well known in the art. PCR thermocycling is commonly employed, and can be used with the presently disclosed methods. PCR techniques are well known in the art, and kits and reagents are commercially available, e.g., from Roche Molecular Systems, Life Technologies, Bio-Rad, etc. In some embodiments, the nucleic acid polymerase is QSU, Taq, or a Taq derivative. A preliminary reverse transcription step can be carried out for RNA targets (also referred to as RT-PCR, not to be confused with real time PCR). See, e.g., Hierro et al. (2006) 72:7148. The term “RT-PCR” as used herein refers to reverse transcription followed by PCR. Both reactions can be carried out in a single tube without interruption, e.g., to add reagents.

Isothermal amplification methods can be employed in the presently described detection methods. Examples of such methods include, without limitation, loop-mediated isothermal amplification (LAMP) (Segawa et al. (2014) J Virol. Methods 201:31-37; Ushio et al. (2005) J. Med. Virol. 77:121), recombinase polymerase amplification (RPA)(Piepenburg et al. (2006) PLoS Biology 4:e204; Euler et al. (2013) J Clin Microbio/51:1110), self-sustained sequence replication (3SR) (Mueller et al. Histochem. Cell Biol. 108:431), strand displacement amplification (SDA) (Walker et al. (1996) Nucl. Acids Res. 24:348), or smart amplification process version 2 (SMAP 2) (Tatsumi et al. (2008) J. Mol. Diagn. 10:520). Isothermal RNA based amplification methods can also be used, e.g., RT-LAMP (Segawa, supra), RT-RPA (Piepenburg, Euler, supra), transcription mediated amplification (TMA) or nucleic acid sequence-based amplification (NASBA). See, e.g., Fakruddin et al. (2013) J Pharm Bioallied Sci. 5:245; van Deursen et al. (1999) Nucl. Acids Res. 27:e15; Kamisango et al. (1999) J Clin. Microbial 37:310.

Isothermal amplification methods can conveniently be used to amplify target nucleic acids in the same vessel with reagents for thermotolerant Cas12 cleavage and detection. Depending on the Cas12 enzyme used and design of the reporter oligonucleotide, an appropriate amplification method can be selected. For example, LAMP is ideally carried out at about 50-65° C., and RPA is ideally carried out at 37-42° C. In some embodiments, the nucleic acid polymerase is Bst2.0 (e.g., Warmstart from NEB) and optionally RTx (e.g., Warmstart RTx from NEB). A number of other appropriate polymerases for isothermal amplification are known and commercially available.

### Cas12

The presently described methods rely on the collateral cleavage activity of the Cas12 enzyme. Any Cas12 enzyme can be used, e.g., a Cas12 protein such as Cas12a, Cas12b, Cas12c, Cas12d, or Cas12e. Cas12 is activated by a guide RNA, which occurs when the sample includes a target DNA sequence to which the guide RNA hybridizes. Upon hybridization, Cas12 is activated and non-specifically cleaves ssDNAs (including non-target ssDNAs) present in the sample. See, e.g., WO2019104058. Particular examples of Cas12 that can be used are AapCas12a, AacCas12b, CmeCas12a, FnCas12a, Engen® LbaCas12a (NEB), or YmeCas12a. In some embodiments, the Cas12 enzyme is thermotolerant, e.g., retaining activity at 45 C-65 C. These include AapCas12a, AacCas12b, YmeCas12a, CmeCas12a, and Engen® LbaCas12a (NEB).

### Detection Reagents

The presently described Cas12 reporter oligonucleotides are designed to emit a fluorescent signal upon collateral cleavage by Cas12 enzyme once the enzyme is bound to the target sequence.

This is accomplished by including a fluorophore at one end of the Cas12 reporter oligonucleotide and a quencher at the other, and optionally also using a quencher that is placed internally in the reporter oligonucleotide, such that the quencher(s) and fluorophore are in sufficient proximity to avoid fluorescence. Typically efficient quenching occurs as long as the fluorophore and quencher are about 6-10, or 6-15 nucleotides apart. Background fluorescence generally starts to become problematic at about 15 nucleotides (50 angstroms or above).

Certain Cas12 enzymes preferentially carry out collateral cleavage of longer oligonucleotides, e.g., over 25 nucleotides in length. To ensure that the fluorophore and quencher remain in proximity, and avoid background fluorescence in the absence of Cas12 target binding, the Cas12 reporter oligonucleotide is designed to have secondary structure bringing the fluorophore and quencher molecule into proximity. This can include a hairpin structure, e.g., having at least 4, 5, 6, 7, 8, or more base pairs.

In some embodiments, the Cas12 reporter oligonucleotide has Cas12-cleavable regions that are primarily outside the hybridized (hairpin) region. In some embodiments, these are the arm(s) and/or loop of an arm-stem-loop-stem-arm structure. In some embodiments, a fluorophore is attached to the end of one arm and a quencher is attached to the end of the other arm. In some embodiments, an internal quencher is used, and the Cas12 reporter oligonucleotide is designed such that the quencher is close to the fluorophore before cleavage and separated from the fluorophore after cleavage by Cas12. For example, the internal quencher can be on the same arm as the end quencher, and the fluorophore at the end of the other arm, so that cleavage will separate both quenchers from the fluorophore.

Any fluorophore (dye) can be used in the presently described methods to label a nucleic acid as described herein. Fluorophores can be attached by conventional covalent bonding, using appropriate functional groups on the fluorophore and/or nucleic acid.

The following are examples of fluorophores that can be used as labels: 4-acetamido-4′-isothiocyanatostilbene-2,2′disulfonic acid; acridine; acridine isothiocyanate; 5-(2′-aminoethypaminonaphthalene-1-sulfonic acid (EDANS); 4-amino-N-[3-vinylsulfonyl)phenyl]naphthalimide-3,5 disulfonate N-(4-anilino-1-naphthyl)maleimide; anthranilamide; BODIPY; Brilliant Yellow; coumarin; 7-amino-4-methylcoumarin (AMC, Coumarin 120)/7-amino-4-trifluoromethylcoumarin (Coumaran 151); cyanine dyes; cyanosine 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′ diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansykhloride); 4-(4′-dimethylaminophenylazo)benzoic acid (DABCYL); 4-dimethylaminophenylazophenyl-4′-isothiocyanate (DABITC); eosin; eosin isothiocyanate; erythrosin B; erythrosin isothiocyanate; ethidium; 6-carboxyfluorescein (FAM); 5-(4,6-dichlorotriazin-2-yl)aminofluorescein (DTAF); 2′,7′-dimethoxy-4′,5′-dichloro-6-carboxyfluorescein (JOE); fluorescein; fluorescein isothiocyanate; fluorescamine; IR144; IR1446; Malachite Green isothiocyanate; 4-methylumbeLiferone; ortho cresolphthalein; nitrotyrosine; pararosaniline; Phenol Red; phycoerythrin (including but not limited to B and R types); o-phthaldialdehyde; pyrene; pyrene butyrate; succinimidyl 1-pyrene butyrate; quantum dots; Reactive Red 4 (Cibacron Brilliant Red 3B-A); 6-carboxy-X-rhodamine (ROX); 6-carboxyrhodamine (R6G); lissamine rhodamine B sulfonyl chloride rhodamine; rhodamine B; rhodamine 123; rhodamine X isothiocyanate; sulforhodamme B; sulforhodamine 101; sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; Alexa 647; and lanthanide chelate derivatives.

A dual labelled reporter is used for detection. The dual labelled reporter can comprise a fluorophore, such any of the fluorophores listed above, and a quencher. Suitable quenchers include but are not limited to DDQ-I, Dabcyl, Eclipse, Iowa Black FQ, BHQ-1, QSY-7, BHQ-2, DDQ-II. Iowa Black RQ, QSY-21, ZEN, and BHQ-3. For fluorophores having an emission maximum between 500 and 550 nm (e.g., FAM, TET, and HEX), a quencher with an absorption maxima between 450 and 500 nm can be selected (e.g., dabcyl or BHQ-1). For fluorophores having an emission maximum above 550 nm (e.g., rhodamine and Cy dyes), a quencher with an absorption maxima above 550 nm can be selected (e.g., BHQ-2). See, e.g., Johansson (2003) Meth. Mol. Biol. 335:17 for considerations in selecting dye-quencher pairs. In some embodiments, the ZEN/Iowa Black FQ quencher pair is used for an internal quencher. In this case, the ZEN quencher can be placed about 6-10 nucleotides (or an equivalent distance in Angstroms) away from the fluorophore, and the IBFQ quencher is attached to the end of the other arm from the fluorophore. See, e.g., Xia et al. (2016) Biotechniques 60:28-33.

FRET technology (see, for example, U.S. Pat. Nos. 5,849,489 and 6,162,603) can also be used in the oligonucleotides disclosed herein. FRET is based on a concept that when a donor fluorescent moiety and a corresponding acceptor fluorescent moiety (or quencher) are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. The terms FRET pair, fluorophore/quencher pair, donor/acceptor pair and like terms are used interchangeably herein.

In some cases (e.g., when the reporter oligonucleotide includes a FRET pair) the reporter oligonucleotide produces an amount of detectable signal prior to being cleaved, and the detectable signal that is measured is different (increased, or a different wavelength) when the reporter oligonucleotide is cleaved. In some cases, the FRET pair produces a first detectable signal prior to being cleaved (e.g., background) and a second detectable signal when the reporter oligonucleotide is cleaved (e.g., once the quencher is distanced from the fluorophore).

An oligonucleotide reporter as disclosed herein can contain a donor fluorescent moiety and a corresponding acceptor fluorophore (or quencher), which dissipates the transferred energy in a form other than light. When the reporter oligonucleotide has appropriate secondary structure, energy transfer typically occurs between the two fluorescent moieties such that fluorescent emission from the donor fluorescent moiety is quenched. Upon cleavage of the reporter oligonucleotide, the donor fluorescent moiety is separated from the quencher and the donor fluorophore can be detected.

As used herein with respect to donor and corresponding acceptor fluorescent moieties “corresponding” refers to an acceptor fluorescent moiety having an emission spectrum that overlaps the excitation spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Donor and acceptor fluorescent moieties can be obtained, for example, from Molecular Probes (Junction City, Oregon, USA) or Sigma Chemical Co. (St. Louis, Missouri, USA).

Frequently used linkers to couple a fluorescent or quencher moiety to an oligonucleotide include thiourea linkers and amide-linkers.

### Measuring a Detectable Signal

The present invention provides methods, kits, and compositions for detecting the presence or absence of a target nucleic acid sequence using the ability of Cas12 to cleave a dual labelled reporter oligonucleotide, once activated.

In some cases, a detectable signal is produced when the reporter oligonucleotide is cleaved (e.g., in some cases, the reporter oligonucleotide comprises a quencher/fluorophore pair). For example, in some cases, an amount of detectable signal increases when the reporter oligonucleotide is cleaved. For example, in the presence of the target nucleic acid sequence, binding of the Cas12 enzyme to the target sequence initiates collateral cleavage of the Cas12 reporter oligonucleotides by the Cas12 enzyme, thereby emitting a fluorescent signal. In the absence of the target nucleic acid sequence, little or no fluorescence will be detected, because the secondary structure of the oligonucleotide holds the fluorophore and quencher in proximity, eliminating the ability of the fluorophore to emit fluorescence.

A quencher moiety can quench a signal from the fluorophore moiety to various degrees (e.g., prior to cleavage of the reporter oligonucleotide by Cas12). In some cases, a quencher moiety quenches the signal from the fluorophore moiety where the signal detected in the presence of the quencher moiety (when the quencher and fluorophore are in proximity to one another) is 25% or less of the signal detected in the absence of the quencher moiety (when the quencher and fluorophore are separated). For example, in some cases, the signal detected in the presence of the quencher moiety can be 30% or less, 20% or less, 15% or less, 10% or less, or 5% or less of the signal detected in the absence of the quencher moiety. In some cases, no signal (e.g., above background) is detected in the presence of the quencher moiety.

In some cases, the signal detected in the absence of the quencher moiety (when the quencher and fluorophore are separated) is at least 1.2 fold greater (e.g., at least 1.3 fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater) than the signal detected in the presence of the quencher moiety (when the quencher and fluorophore are in proximity to one another).

In some embodiments, the present methods disclosed herein include a step of measuring a detectable signal produced by Cas12 enzyme-mediated cleavage of the reporter oligonucleotide.

The measuring can in some cases be qualitative, e.g., in the sense that the presence or absence of detectable signal can indicate the presence or absence of the target nucleic acid sequence. The measuring can in some cases be quantitative, e.g., in the sense that the amount of signal detected can be used to determine the amount of target nucleic acid present in the sample. In some cases, a detectable signal will not be present (e.g., above a given threshold level) unless the target nucleic acid sequence is present above a particular threshold concentration.

The measuring can be done by any fluorescence detection method known in the art, for example a microplate reader, fluorescence spectrophotometer, or by a mobile phone reader.

### Kits

Provided herein are kits for carrying out the presently described detection methods. In some embodiments, the kit comprises reagents for the Cas12 reaction, e.g. a labelled Cas12 reporter oligonucleotide. In some embodiments, the kit further comprises a Cas12 enzyme (e.g., a thermotolerant Cas12 enzyme such as CmeCas12a), and an appropriate buffer. In some embodiments, the kit further comprises a positive control, e.g., a known target nucleic acid and matching guide RNA.

In some embodiments, the kit comprises reagents for an amplification reaction such as a nucleic acid polymerase, nucleotides, and/or a buffer. In some embodiments, the amplification reaction is isothermal.

In some embodiments, the kit is designed to be specific for a particular target nucleic acid, and includes primers and/or a guide RNA specific for that target nucleic acid, and/or a positive control nucleic acid.

The kit can also include consumables for the reactions, e.g., a multiwell plate and/or vessels for mixing reagents. The kit can also include instructions, or directions to an appropriate database for obtaining and/or processing data.

### EXAMPLES

### Example 1: Identify a ssDNA Molecule to Report the Collateral Cleavage Activity of Cas12a Upon its Activation by a Target dsDNA

We have found that the standard ssDNA (5′-6FAM-TTATT-IwBKQ-3′, SEQ ID NO:1) used to report the collateral cleavage activity of LbaCas12a is not cleaved by this enzyme or the CmeCas12a enzyme when the reactions are performed at temperatures at or above 45° C. In addition to this temperature effect we also found that the sequence of the reporter oligonucleotide could have a marked impact on the effectiveness of the reporter. The observation of Cas enzyme collateral cleavage sequence bias was previously reported in a study of Cas13 orthologues Gootenberg et al. 2018 supra found that while all Cas13 enzymes were able to cleave M13 ssDNA into very small fragments, each isoform showed distinct preferences for certain dinucleotide motifs. However, most of the enzymes tested could cleave more than one dinucleotide.

Producing each dinucleotide pair labelled with fluorophore and quencher is an expensive and slow process. Furthermore preliminary experiments suggested that small oligonucleotides (N>10) would likely be poor substrates for collateral cleavage by LbaCas12a and CmeCas12a, and potentially other thermotolerant Cas12a enzymes, at the relevant temperatures.

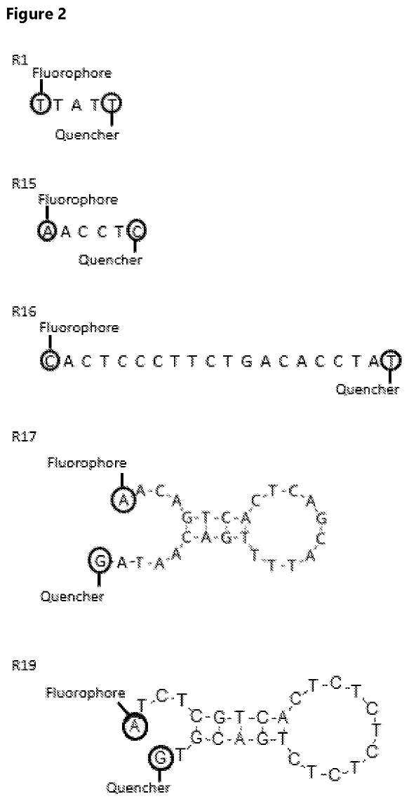

We used the various fluorophore-quencher labelled ssDNA oligonucleotides shown in Table 2 to determine the extent of the collateral cleavage activity by CmeCas12a. Each oligonucleotide was labelled with 6-FAM at the 5′ end and BHQ-1 at the 3′ end. The sequence varies between the oligonucleotides with a useful diversity of dinucleotide prevalence. We also noted that some of the oligonucleotides in our collection were predicted to form secondary structure at the target temperatures (FIG. 2).

All oligonucleotides were resuspended in water at a concentration of 100 μM and stored in −20° C. in the dark. Working stocks of each oligonucleotide were diluted from these stocks using water to a final concentration of 1 μM, again these were stored at −20° C. in the dark.

TABLE 2
Sequences of reporter oligonucleotides

| | | | SEQ |
|---|---|---|---|
| | | | ID |
| Name | Length | Sequence | NO |
| R1 | 5 | TTATT | 1 |
| R2 | 22 | CAAGGAGCTCGTCGTCATTTTC | 2 |
| R3 | 28 | ACTCCTCTCAGCTCTCATTCCTATCAAC | 3 |
| R4 | 27 | AAGATGAGCAAGGCGTGTAGGTGTGGC | 4 |
| R5 | 30 | CGTCCCTTCTTCTTTACCTTCTCCTGG | 5 |
| | | CTC | |
| R6 | 25 | CGTGTCACCAGCCATTGAGTGTCAC | 6 |
| R7 | 25 | CCCCTGGAAGCATCGCTGGTITTAT | 7 |
| R8 | 28 | AAGCAGCTTGAAGACGGTCGAACTCTGG | 8 |
| R9 | 29 | AAGAGTCACTCACTTTTGACAGTTATACG | 9 |
| R10 | 25 | ATGTGTAGGGCACAGAAGTCACCTG | 10 |
| R11 | 27 | CGCATGTTATCACAGGAAGTCGTGTTG | 11 |
| R12 | 26 | TGTCAACACTCTCCCAGGTAGTAACG | 12 |
| R13 | 26 | TCCATCGCCACCTAATTCTCTTTCCT | 13 |
| R14 | 26 | GGTACAAATCTCAGGTCCCTATTGGA | 14 |
| R15 | 6 | AACCTC | 15 |
| R16 | 20 | CACTCCCTTCTGACACTTAT | 16 |
| R17 | 27 | AACAGTCACTCAGCATTTTGACAATAG | 17 |

The CmeCas12a enzyme was supplied by NEB in 1× storage buffer with glycerol at a concentration of 440 μM. The enzyme was diluted into 1× storage buffer at a final concentration of 1 μM without glycerol. This stock was stored at 4° C. for up to 2 weeks prior to use.

The guide RNA oligonucleotides used in this Example are of the general form recommended by the supplier (IDT) for LbaCas12a. The particular RNA guides used in this study were designed to detect dsDNA copies of the N-gene and E-gene of the SARS-Cov2 virus. These oligonucleotides are made with native ribonucleotides and have not been modified with any protective chemical motifs.

E-gene guide RNA
(SEQ ID NO: 18)
UAAUUUCUACUAAGUGUAGAUCCCCCAGCGCUUCAGCGUUC
N-gene guide RNA
(SEQ ID NO: 19)
UAAUUUCUACUAAGUGUAGAUGUGGUAUUCUUGCUAGUUAC

Using SARS-Cov2 reference RNA supplied by Twist Biosciences, we produced cDNA using Superscript IV (Invitrogen) using gene specific primers for the E and N gene (Broughton et al. (2020) Nat. Biotechnol. 38:870-874) target loci following the manufacturer's instructions.

TABLE 3
E-gene and N-gene primer sequences

| | | SEQ ID |
|---|---|---|
| Primer name | Primer sequence | NO |
| E-gene for | CCGACGACGACTACTAGC | 20 |
| E-gene rev | AGAGTAAACGTAAAAAGAAGGTT | 21 |
| N-gene for | AACACAAGCTTTCGGCAG | 22 |
| N-gene rev | GAAATTTGGATCTTTGTCATCC | 23 |

Standardised dsDNA targets for the N-gene and E-gene containing dUracil were produced from this cDNA for testing with Cas12a. While dTTP can be used alone, we included a 50-50 mixture of dUTP and dTTP to avoid contamination from previously amplified product. We have previously shown that the presence of dUracil in dsDNA target does not prevent the recognition of the dsDNA by LbaCas12a or CmeCas12a. We used Q5U Hifi polymerase (NEB) and the same primers as for the reverse transcription reaction following the manufacturer's instructions. The nucleotide mix contained equal amounts of dTTP and dUTP. The resulting DNA was purified by column (Macherey-Nagel) and quantified by spectroscopy (Nanodrop) before being diluted to 1 ng/μL and stored at 4° C.

CmeCas12a was mixed in an equimolar ratio with the appropriate guide RNA and reporter at a final concentration of 100 nM in 1×NEB2.1 buffer, leaving out the target DNA. The master mixes were incubated at 37° C. for 30 minutes, before being cooled on ice. Each of the two target DNAs (5 ng) and the matching no template controls were pipetted into a 384-well plate in triplicate for each Cas12 reporter/guide pair and cooled on ice. The final reaction volume was 20 μL. The preincubated master mixes were added to the DNA or NTC containing wells and sealed with ultraclear film, mixed thoroughly, briefly centrifuged, and returned to ice.

A BMG Omega FluoStar fluorescence plate reader was prewarmed to 45° C. The excitation and emission filters were 480 nm and 520 nm respectively. The plate was moved directly from ice to the plate reader and was incubated at 45° C. with the fluorescence measured at 2 minute intervals for 31 readings. The plate was returned to ice. The fluorescence data was exported to Excel for further analysis.

To ensure that each reaction was viable, we further treated each reaction with 2 units of Mung bean nuclease (NEB) for 16 hours at 30° C. in a final volume of 25 μL. The fluorescence of the reactions was measured at 45° C. as above.

Excel was used to sort and label the data before it was imported to Prizm v 9 where means and SD were calculated and graphed.

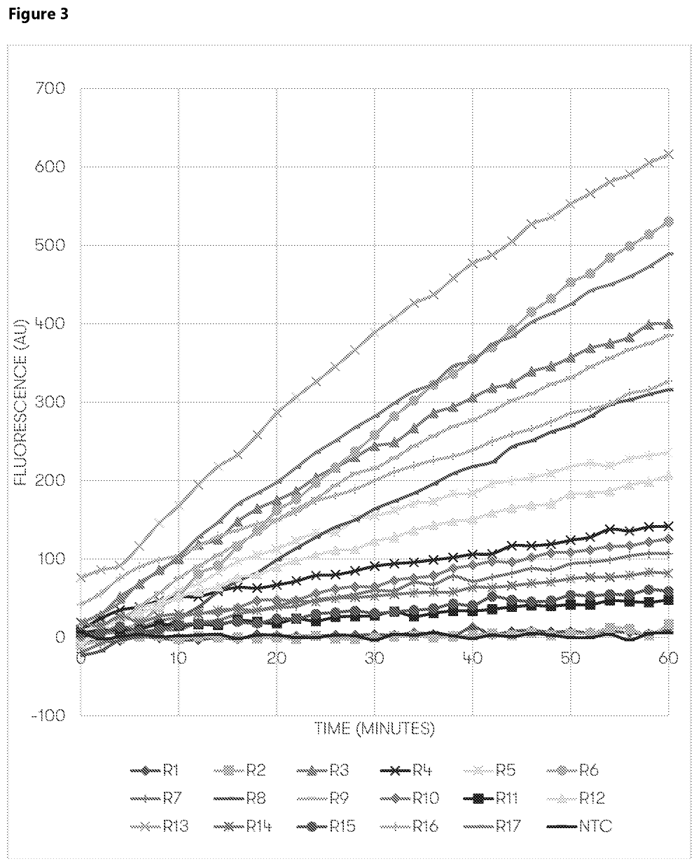

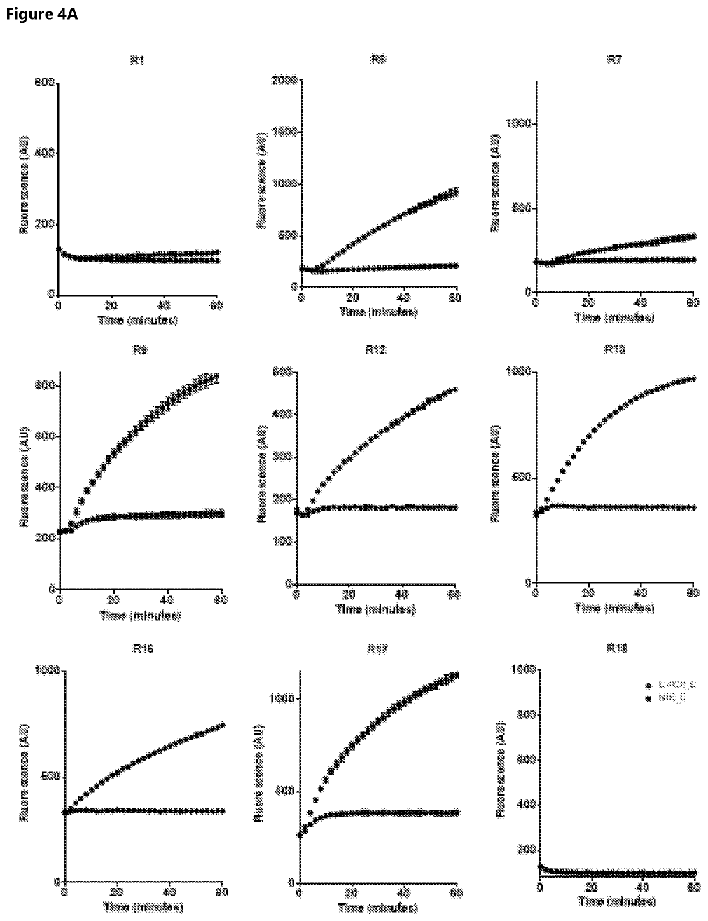

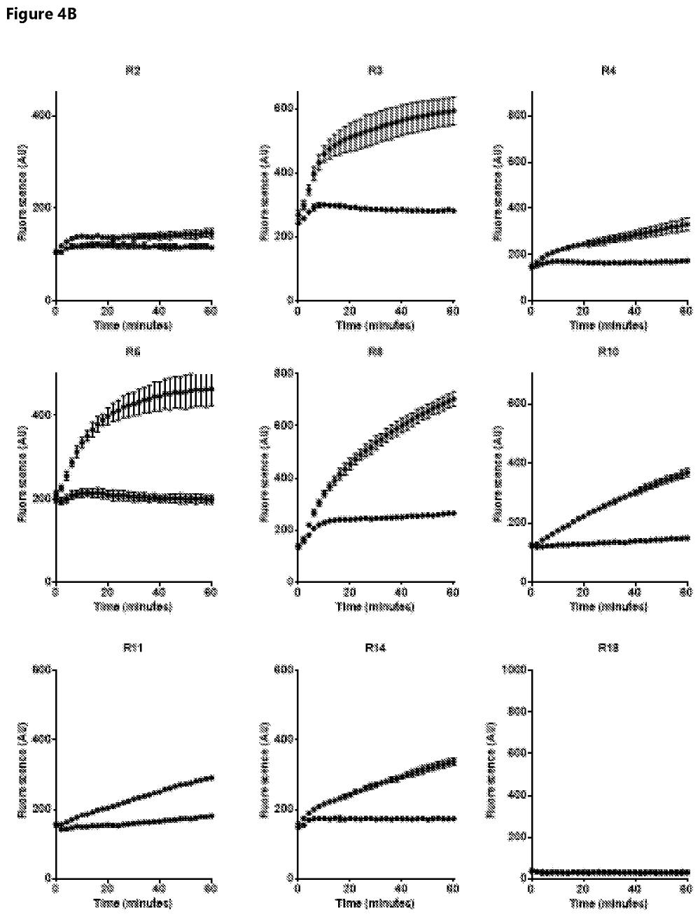

FIG. 3 shows the NTC subtracted fluorescence data for each oligonucleotide reporter reaction during the 60 minute incubation with CmeCas12a and the E-gene target/guide pair. FIGS. 4A and 4B show each reporters reaction raw fluorescence data scaled to the MBN result during the 60 minute incubation.

The table below (Table 4) shows the Cas12 collateral cleavage activity for each reporter oligonucleotide expressed in percentage cleaved at 16 and 60 minutes of the reactions shown in FIG. 4. The remaining columns list the number of each dinucleotide pair present in that oligonucleotide.

The results indicate that longer reporter oligonucleotides are cleaved at a higher rate, and that the CmeCas12a enzyme preferentially cleaves those with CT and TC dinucleotides in the single stranded regions of the DNA. We observed similar results for the N-gene guide/target pairs showing they were able to produce collateral cleavage by Cas12, demonstrating that this system can be used to detect any target sequence.

TABLE 4
Percent cleavage and dinucleotide rates in certain reporter oligonucleotides

| | Activity | (% cleaved) | Numbers of dinucleotides present | | | | | | | | | | | | | | | |
|---|---|---|---|---|---|---|---|---|---|---|---|---|---|---|---|---|---|---|
| Name | 16 min | 60 min | CT | TC | CA | TT | AC | CC | AA | TA | AT | TG | AG | GA | GC | CG | GG | GT |
| R1 | 1 | 5 | | | | 2 | | | | 1 | 1 | | | | | | | |
| R2 | 6 | 12 | | 4 | 2 | 1 | | | 1 | | 1 | | 2 | 1 | 1 | 2 | 1 | 2 |
| R3 | 77 | 100 | 6 | 7 | 3 | 1 | 1 | 2 | 1 | 1 | 2 | | 1 | | 1 | | | |
| R4 | 10 | 24 | | | 1 | | | | 2 | 1 | 1 | 4 | 4 | 2 | 3 | | 3 | 4 |
| R5 | 78 | 100 | 7 | 6 | | 5 | 1 | 4 | | 1 | | 1 | | | 1 | 1 | 1 | 1 |
| R6 | 10 | 41 | | 2 | 4 | 1 | 2 | 2 | | | 1 | 3 | 1 | 1 | 1 | 1 | | 4 |
| R7 | 4 | 13 | 2 | 1 | 1 | 3 | | 3 | 1 | 1 | 2 | 2 | 1 | 1 | 2 | 1 | 2 | 1 |
| R8 | 40 | 98 | 3 | 2 | 1 | 1 | 2 | | 3 | | | 2 | 3 | 3 | 1 | 2 | 2 | 1 |
| R9 | 37 | 100 | 2 | 2 | 3 | 4 | 4 | | 1 | 2 | 1 | 1 | 1 | 2 | | 1 | | |
| R10 | 14 | 42 | | 1 | 3 | | 2 | 1 | 1 | 1 | 1 | 3 | 3 | 1 | 1 | | 2 | 3 |
| R11 | 12 | 28 | | 2 | 3 | 2 | 1 | | 1 | 1 | 2 | 3 | 2 | 1 | 1 | 2 | 1 | 4 |
| R12 | 36 | 100 | 2 | 3 | 3 | | 2 | 2 | 2 | 2 | | 1 | 2 | | | 1 | 1 | 3 |
| R13 | 55 | 100 | 4 | 5 | 3 | 3 | 1 | 4 | 1 | 1 | 2 | | | | 1 | 1 | | |
| R14 | nd | nd | 2 | 3 | 2 | 1 | 1 | 2 | 2 | 2 | 2 | 1 | 1 | 1 | | | 3 | 2 |
| R15 | 7 | 20 | 1 | 1 | | | 1 | 1 | 1 | | | | | | | | | |
| R16 | 25 | 67 | 4 | 2 | 2 | 2 | 2 | 2 | | 1 | 1 | 1 | | 1 | | | | |
| R17 | 42 | 100 | 1 | 2 | 5 | 3 | 3 | | 2 | 1 | 2 | 1 | 2 | 1 | 1 | | | 1 |

### Example 2: Confirm that the Target Sequence does not Affect Collateral Cleavage of the Reporter Oligonucleotide by CmeCas12a

We sought to confirm that Cas12 can be used to target any sequence and result in collateral cleavage of the Cas12 variant's reporter oligonucleotide, using the E-gene and N-gene as target nucleic acids.

As in the previous example we produced standardised template DNA for the targets which contained both dTTP and dUTP.

We next set up the Cas12 cleavage reactions as follows using 8 of the reporter oligonucleotides described above, and 1 reaction without reporter.

### Reaction mix (20 uL):

| Reagent | Concentration | |
|---|---|---|
| DNA target | 5 | ng |
| NEB2.1 buffer | 1× | |
| CmeCas12a | 100 | nM |
| Guide RNA | 100 | nM |
| Reporter oligonucleotide (see Table 5) | 100 | nM |

Water

Each reaction was carried out in triplicate. The mixtures were incubated at 37 C without the E-gene or N-gene target, cooled on ice, and added to target DNA in a 384 well plate. Reactions were carried out as in Example 1, with readings every 2 minutes for 31 readings. At the end of each reaction, we again added Mung Bean Nuclease to confirm the viability of each reaction. The endpoint florescence results are shown in Table 5 below.

TABLE 5
Fluorescence data for CmeCas12a reactions with certain reporter
oligonucleotides

| | | | | % cleavage |
|---|---|---|---|---|
| Reporter | CmeCas12a | CmeCas12a | Mung bean | (ΔCme/ |
| name | minimum | maximum | nuclease max. | ΔMBN) |
| R1 | 114 | 120 | 700 | 1 |
| R6 | 165 | 930 | 2000 | 41 |
| R7 | 180 | 320 | 1250 | 13 |
| R9 | 280 | 850 | 850 | 100 |
| R12 | 175 | 450 | 450 | 100 |
| R13 | 380 | 970 | 970 | 100 |
| R16 | 340 | 740 | 940 | 67 |
| R17 | 390 | 1150 | 1150 | 100 |
| No reporter | — | — | — | — |

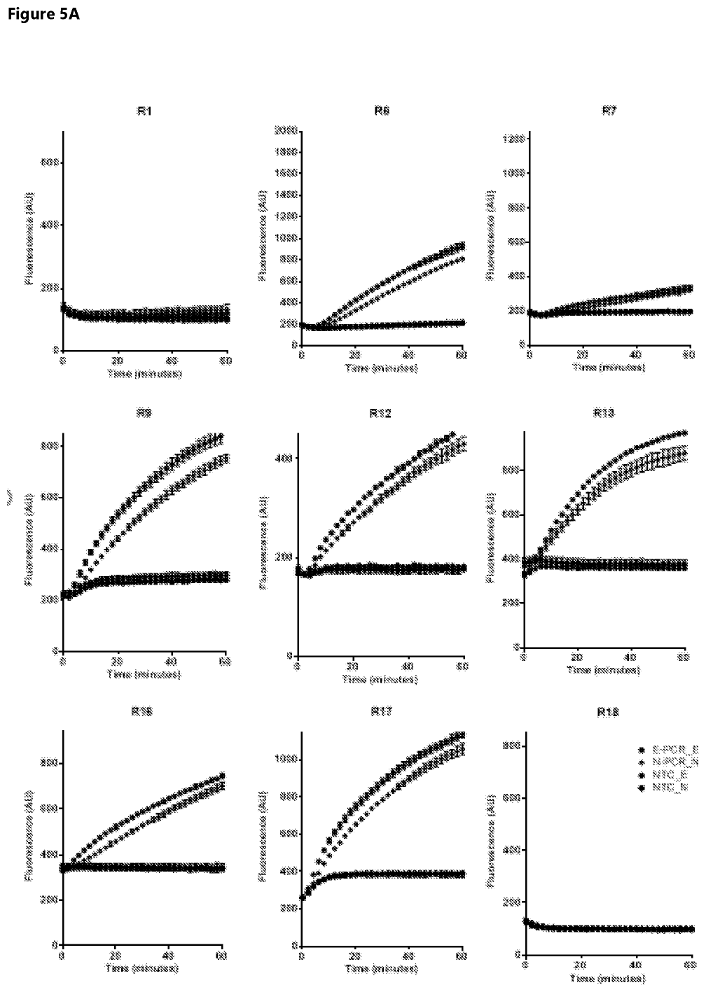

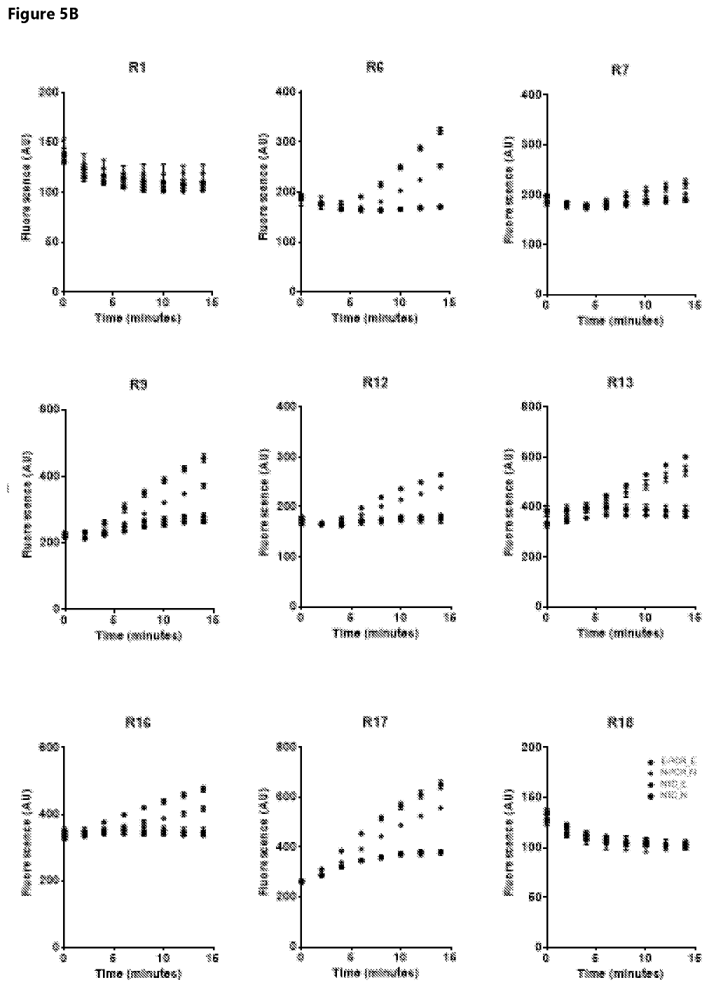

As shown in FIGS. 5A (full 60 minute time course) and 5B (truncated 15 minutes time course to show initial rate of reaction), we observed similar results for both E-gene and N-gene targets, demonstrating that the present methods are able to detect any target sequence.

### Example 3: Modelling for Additional Reporter Oligonucleotides

We designed additional Cas12 reporter oligonucleotides in part to expand the potential temperature range available for enzymatic reactions. For example, if LAMP or RT-LAMP is used for amplification, the commonly used enzymes are generally more efficient at 55-65 C. To maintain the hairpin structure of the reporter oligonucleotide, we sought to expand the length of the hybridized stem region to 5 or 6 base pairs. As is evident from the sequences below, CT dinucleotides are included to maximize CmeCas12a cleavage. Exemplary oligonucleotide sequences are shown below.

TABLE 6
Additional Cas12a reporter
oligonucleotide sequences

| | | SEQ |
|---|---|---|
| | | ID |
| Name | Sequence | NO |
| R19 | ATCTCGTCACTCTCTCTCTCTGACGTG | 24 |
| R20 | AACGTCACTCTCTCTCTCTCTTGACGAG | 25 |
| R21 | CTCTCTCTCTAGTCACTGAATGACTTTA | 26 |
| R22 | CTCTCTCTCTAGTCACATGAGTGACTT | 27 |
| R23 | ATATATATATCGTCAATGAATGACGTC | 28 |

The original R1 reporter oligonucleotide is consists of TT, AT and TA dinucleotides. It is the nature of our reporter design that TA and AT dinucleotides cannot be included in a repeated motif within the loop region, as this would cause additional secondary structure to form that would likely prevent the collateral cleavage activity of Cas12 enzymes. R23 was designed to be a reporter oligonucleotide for LbaCas12a which we expected would cleave AT dinucleotides more efficiently. R21 and R22 can be adapted similarly by switching the CT dinucleotides with AT dinucleotides.

Table 7 shows the length of the hairpin, and the highest temperature that the hairpin structure is predicted to remain at least partially intact for each reporter oligonucleotide.

TABLE 7
Characteristics of the R19-R23 reporter oligonucleotides

| Name | Length of hairpin (bp) | Maximum temperature (C.) |
|---|---|---|
| R19 | 5 | 58 |
| R20 | 5 | 55 |
| R21 | 5 | 62 |
| R22 | 6 | 72 |
| R23 | 5 | 71 |

### Example 4: Show that R19-Type Reporter Molecules can be Used Effectively with LbaCas12a in 45° C. Reactions to Detect Multiple Target Sequences

LbaCas12a is most commonly used at 37° C. where it is an effective component of CRISPR-Dx diagnostic systems when combined with the original R1 reporter molecule. As part of our work exploring the temperature sensitivity of Cas12a enzymes we found that LbaCas12a did not cleave the R1 reporter in collateral cleavage reactions when the temperature is maintained at 45° C. However, it is still an effective enzyme at 45° C. with a suitable thermotolerant reporter molecule such as the longer reporters R2-R14 and R16-R23.

The methods used in this Example are largely the same as in Example 1 but with the following modifications.

The LbaCas12a enzyme was supplied by NEB in 1× storage buffer with glycerol at a concentration of 100 μM. The enzyme was diluted into 1×NEB2.1 buffer at a final concentration of 1 μM prior to use. This stock was stored at 4° C. for up to 1 day prior to use.

We used three target DNA molecules. These were the E-gene and N-gene (see Example 1) as well as the A-gene target. The A-gene target DNA was created from the Orf1ab reading frame of the SARS-Cov2 viral RNA in the same way as the other two targets. The primers used to reverse transcribe and create the standardised DNA template are shown in Table 8 (Rabe and Cepko (2020) PNAS 39:24450-24458). To examine the limit of detection using LbaCas12a to detect these target DNAs we created a dilution series of each DNA from 440 μM to 5.5 μM by serial dilution. A neutral carrier DNA was added to all the dilutions to ensure the target DNA was stable in solution.

TABLE 8
Sequences of A-gene primers

| | | SEQ |
|---|---|---|
| | | ID |
| Primer name | Primer sequence | NO |
| A-gene for | CGGTGGACAAATTGTCAC | 29 |
| A-gene rev | CTTCTCTGGATTTAACACACTT | 30 |

The A-gene guide RNA was produced at IDT (Singapore) and is Alt-R modified.

(SEQ ID NO: 31)
UAAUUUCUACUAAGUGUAGAUUUUGUGUGCUGACUCUAUCAUUAU

We produced the R19 reporter with a 5′-6′FAM fluorophore, a 3′-IwBKQ quencher and an internal ZEN quencher on position 25 of the oligonucleotide. In this Example the reporter concentration was increased to 200 nM in the final reaction.

To better control the reaction temperature and achieve the target incubation temperature of 45° C. as quickly as possible we employed an Analytic Jena Q-tower3 qPCR machine to heat the samples and record the fluorescence. Due to the nature of the machine, fluorescence readings are made at t=0 and thereafter once per cycle, where each cycle is approximately 1 minute. The raw fluorescence data was exported from the software to excel file, sorted, and then graphed in Prizm v9.

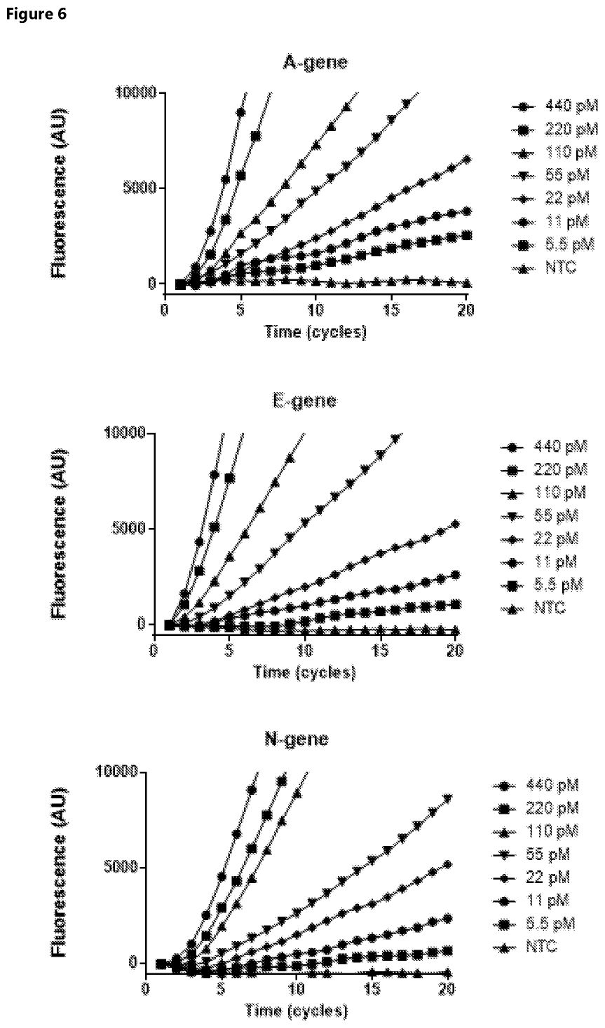

Our results show that the three target genes are detected rapidly and accurately by the combination of LbaCas12a with the correct guide RNA and the R19 reporter molecule (FIG. 6). The sensitivity of the reaction is such that using this modality of detection the target DNA at a concentration of 5.5 μM is reliably detected in less than 15 minutes. All three target genes are detected with similar sensitivity, suggesting there is no effect of the target sequence on the effectiveness of the reporter.

### Example 5: Confirm that the LbaCas12a Enzyme has Limited Dinucleotide Preference

During the experiments for Example 4 we found that the LbaCas12a enzyme was quite capable of efficiently cleaving the R19 reporter molecule, despite there being only a single AT dinucleotide in the molecule. To further test the dinucleotide preference of the LbaCas12a enzyme we produced three R19 variants: R19-CA, R19-GT and R19-GA; in this naming scheme R19 would more expansively be called R19-CT (see Table 9).

TABLE 9
R19-type Cas12a reporter
oligonucleotide sequences

| | | SEQ |
|---|---|---|
| | | ID |
| Name | Sequence | NO |
| R19 (-CT) | ATCTCGTCACTCTCTCTCTCTGACGTG | 24 |
| R19-CA | TACTCGTCACACACACACACTGACGTG | 32 |
| R19-GT | ATGTCGTCAGTGTGTGTGTGTGACGTG | 33 |
| R19-GA | TAGTCGTCAGAGAGAGAGAGTGACGTG | 34 |

The methods were similar to those in Example 1 with the following modifications.

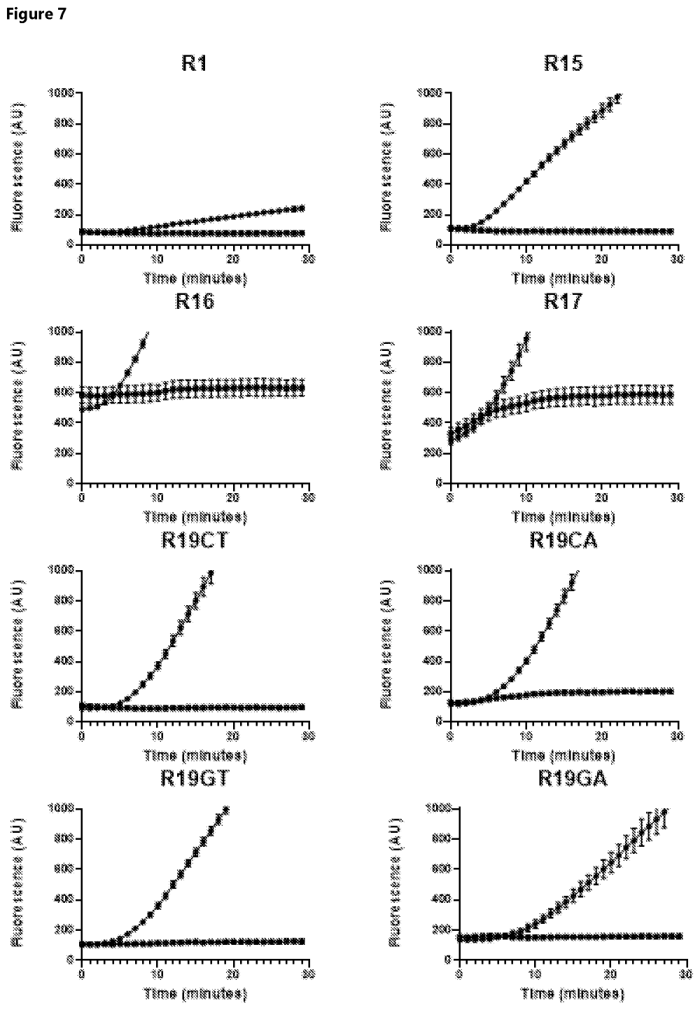

The N-gene target molecule was created, standardised and used as previously described. The unmodified N-gene guide was used. The LbaCas12a was used as described in Example 4. We tested the four R19-type reporter molecules along with the R1, R15, R16 and R17 molecules for comparison with and without target DNA. The concentration of the reporter was 200 nM in the final reaction. Each reaction was performed in triplicate at 45° C. and the data recorded using the Omega Fluostar plate reader. Excel was used to sort and label the data before it was imported to Prizm v9 where means and SD were calculated and graphed (FIG. 7).

LbaCas12a collateral cleavage at 45° C. of the R1 reporter was very poor. The cleavage of the R19, R19-CA and R19-GT reporter oligonucleotides is efficient and produces very low background fluorescence. However, the R19-GA reporter molecule is less effective with its rate of cleavage being similar to that of the R15 reporter. The R16 and R17 reporter molecules were cleaved at a very high rate by LbaCas12a in these conditions. However R16 and R17 reporters produced high background fluorescence in these conditions.

1.-24. (canceled)

25. A Cas12 reporter oligonucleotide comprising:
a) at least 25 nucleotides;
b) an arm-stem-loop-stem-arm structure, wherein:
the arm regions are single-stranded,
the stem regions hybridize to one another, with base pairing extending over 4 or more nucleotides, and
the loop region is single-stranded and greater than 5 nucleotides in length;
c) 2-14 Cas12 preferred dinucleotides, wherein the Cas12 preferred dinucleotides are in the loop and/or arm regions; and
d) a fluorophore molecule on one end of the oligonucleotide and a quencher molecule on the other end;
wherein the oligonucleotide is able to be cleaved by Cas12 enzyme in the presence of a guide RNA bound to a target sequence.

26. The Cas12 reporter oligonucleotide of claim 25, wherein the Cas12 preferred dinucleotides are selected from CT and TC, AT and TA, CA and AC, and GT and TG.

27. The Cas12 reporter oligonucleotide of claim 25, wherein one of the arm regions has 6-14 nucleotides, and the Cas12 preferred dinucleotides are in the arm.

28. The Cas12 reporter oligonucleotide of claim 25, wherein the loop region has 8-14 nucleotides, and the Cas12 preferred dinucleotides are in the loop.

29. The Cas12 reporter oligonucleotide of claim 25, further comprising an internal quencher placed 6-10 nucleotides away from the fluorophore molecule, optionally the internal quencher is ZEN and the quencher is Iowa Black FQ.

30. The Cas12 reporter oligonucleotide of claim 25, wherein the Cas12 enzyme is CmeCas12a, YmeCas12a, or LbaCas12a.

31. The Cas12 reporter oligonucleotide of claim 25, wherein the Cas12 enzyme is:
i) CmeCas12a and the Cas12 preferred dinucleotides are CT and TC; or
ii) LbaCas12a and the Cas12 preferred dinucleotides are AT and TA.

32. The Cas12 reporter oligonucleotide of claim 25, wherein the oligonucleotide comprises:
a) a sequence that is at least 90% identical to a sequence selected from the group consisting of:
(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG;
or
b) a sequence selected from the group consisting of:
(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG,
(R20, SEQ ID NO: 25)
AACGTCACTCTCTCTCTCTCTTGACGAG,
(R21, SEQ ID NO: 26)
CTCTCTCTCTAGTCACTGAATGACTTTA,
(R22, SEQ ID NO: 27)
CTCTCTCTCTAGTCACATGAGTGACTT,
(R23, SEQ ID NO: 28)
ATATATATATCGTCAATGAATGACGTC,
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

33. A kit comprising:
a) the Cas12 reporter oligonucleotide of claim 25;
b) a nucleotide polymerase; and
c) a thermotolerant Cas12 enzyme,
and optionally:
d) oligonucleotide primers specific for a target sequence; and
e) a guide RNA specific for the target sequence.

34. The kit of claim 33, wherein the nucleotide polymerase is:
i) a DNA and/or RNA polymerase;
ii) Bst2.0 and optionally reverse transcriptase; or
iii) Q5U and optionally reverse transcriptase.

35. A method of detecting the presence or absence of a target sequence comprising
a) isolating target nucleic acid;
b) contacting the target nucleic acid with: a nucleotide polymerase, oligonucleotide primers specific for a target sequence on the target nucleic acid, a guide RNA oligonucleotide specific for the target sequence, a thermotolerant Cas12 enzyme, and the Cas12 reporter oligonucleotide of claim 25;
c) incubating at a temperature of about 42 C to about 58 C for at least 15 minutes; and
d) measuring fluorescence, wherein detecting fluorescence produced by cleavage of the oligonucleotide by the Cas12 enzyme is indicative of the presence of the target sequence, and wherein the absence of fluorescence is indicative of the absence of the target sequence.

36. The method of claim 35, wherein the nucleotide polymerase is Bst2.0 polymerase and optionally reverse transcriptase.

37. A method of detecting the presence or absence of a target sequence comprising
a) isolating target nucleic acid;
b) contacting the target nucleic acid with a nucleotide polymerase and oligonucleotide primers specific for a target sequence on the target nucleic acid, and amplifying the target sequence to produce amplified target sequence;
c) contacting the amplified target sequence with a guide RNA oligonucleotide specific for the target sequence, a thermotolerant Cas12 enzyme, and the Cas12 reporter oligonucleotide of claim 25;
d) incubating at a temperature of about 42 C to about 58 C for at least 15 minutes; and
e) measuring fluorescence, wherein detecting fluorescence produced by cleavage of the oligonucleotide by the Cas12 enzyme is indicative of the presence of the target sequence, and wherein the absence of fluorescence is indicative of the absence of the target sequence.

38. The method of claim 37, wherein the nucleotide polymerase is Q5U and optionally reverse transcriptase.

39. The method of claim 35, wherein the thermotolerant Cas12 enzyme is CmeCas12a, YmeCas12a, or LbaCas12a.

40. The method of claim 37, wherein the thermotolerant Cas12 enzyme is CmeCas12a, YmeCas12a, or LbaCas12a.

41. The method of claim 35, wherein the oligonucleotide comprises:
a) a sequence that is at least 90% identical to a sequence selected from the group consisting of:
(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG
(R20, SEQ ID NO: 25)
AACGTCACTCTCTCTCTCTCTTGACGAG,
(R21, SEQ ID NO: 26)
CTCTCTCTCTAGTCACTGAATGACTTTA
(R22, SEQ ID NO: 27)
CTCTCTCTCTAGTCACATGAGTGACTT
(R23, SEQ ID NO: 28)
ATATATATATCGTCAATGAATGACGTC
(R19-CA, SEQ ID NO: 32)
TACTCGTCACACACACACACTGACGTG
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG;
or
b) a sequence selected from the group consisting of:
(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG
(R20, SEQ ID NO: 25)
AACGTCACTCTCTCTCTCTCTTGACGAG,
(R21, SEQ ID NO: 26)
CTCTCTCTCTAGTCACTGAATGACTTTA
(R22, SEQ ID NO: 27)
CTCTCTCTCTAGTCACATGAGTGACTT
(R23, SEQ ID NO: 28)
ATATATATATCGTCAATGAATGACGTC
(R19-CA, SEQ ID NO: 32)
TACTCGTCACACACACACACTGACGTG
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

42. The method of claim 37, wherein the oligonucleotide comprises:
a) a sequence that is at least 90% identical to a sequence selected from the group consisting of:
(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG
(R20, SEQ ID NO: 25)
AACGTCACTCTCTCTCTCTCTTGACGAG,
(R21, SEQ ID NO: 26)
CTCTCTCTCTAGTCACTGAATGACTTTA
(R22, SEQ ID NO: 27)
CTCTCTCTCTAGTCACATGAGTGACTT
(R23, SEQ ID NO: 28)
ATATATATATCGTCAATGAATGACGTC
(R19-CA, SEQ ID NO: 32)
TACTCGTCACACACACACACTGACGTG
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG;
or
b) a sequence selected from the group consisting of:
(R3, SEQ ID NO: 3)
ACTCCTCTCAGCTCTCATTCCTATCAAC,
(R5, SEQ ID NO: 5)
CGTCCCTTCTTCTTTACCTTCTCCTGGCTC,
(R8, SEQ ID NO: 8)
AAGCAGCTTGAAGACGGTCGAACTCTGG,
(R9, SEQ ID NO: 9)
AAGAGTCACTCACTTTTGACAGTTATACG,
(R12, SEQ ID NO: 12)
TGTCAACACTCTCCCAGGTAGTAACG,
(R13, SEQ ID NO: 13)
TCCATCGCCACCTAATTCTCTTTCCT,
(R17, SEQ ID NO: 17)
AACAGTCACTCAGCATTTTGACAATAG,
(R19, SEQ ID NO: 24)
ATCTCGTCACTCTCTCTCTCTGACGTG
(R20, SEQ ID NO: 25)
AACGTCACTCTCTCTCTCTCTTGACGAG,
(R21, SEQ ID NO: 26)
CTCTCTCTCTAGTCACTGAATGACTTTA
(R22, SEQ ID NO: 27)
CTCTCTCTCTAGTCACATGAGTGACTT
(R23, SEQ ID NO: 28)
ATATATATATCGTCAATGAATGACGTC
(R19-CA, SEQ ID NO: 32)
TACTCGTCACACACACACACTGACGTG,
(R19-GT, SEQ ID NO: 33)
ATGTCGTCAGTGTGTGTGTGTGACGTG,
and
(R19-GA, SEQ ID NO: 34)
TAGTCGTCAGAGAGAGAGAGTGACGTG.

43. The method of claim 35, wherein step a) is carried out using enzymatic purification.

44. The method of claim 37, wherein step a) is carried out using enzymatic purification.
